(19) United States
(12) Patent Application Publication
(10) Pub. No.: US 2019/0066838 A1
(43) Pub. Date: Feb. 28, 2019

(54) SURGICAL SCHEDULING SYSTEM

(71) Applicant: SIGMA SURGICAL CORPORATION, New York, NY (US)

(72) Inventors: Evan Garfein, New York, NY (US); Craig Overpeck, Blue Bell, PA (US)

(21) Appl. No.: 16/114,427

(22) Filed: Aug. 28, 2018

Related U.S. Application Data
(60) Provisional application No. 62/551,305, filed on Aug. 29, 2017.

(51) Int. Cl. G16H 40/20; G06Q 10/06

(52) U.S. Cl. CPC G16H 40/20; G06Q 10/063

(57) ABSTRACT

An example of a computer-implemented method includes receiving a surgical protocol for a surgical procedure, a team member identifier for a team member, and team member data associated with a plurality of steps in the surgical protocol. The method further includes determining a number of times that the team member has performed a particular step at an unassisted qualification level. The method further includes determining whether the number of times meets a threshold level. The method further includes, responsive to determining that the number of times does not meet the threshold level, designating the particular step as having an observation requirement by a team leader, and, responsive to determining that the number of times meets the threshold level, designating the particular step as having no observation requirement by the team leader. The plurality of steps in the surgical protocol is then displayed to the team member.

### CROSS-REFERENCE TO RELATED APPLICATIONS

This application claims the benefit of U.S. Provisional Application No. 62/551,305 filed Aug. 29, 2017, entitled “SURGICAL SCHEDULING SYSTEM,” which is incorporated by reference herein in its entirety.

### BACKGROUND

The present invention generally relates to surgical scheduling, and more specifically, to coordinating the tasks performed by surgeons.

In the medical community a surgical team may include a resident or trainee doctor who works under the supervision of a senior surgeon or team leader.

As the resident doctor becomes more proficient performing surgery, some of the steps or tasks of a particular surgery may be performed by the resident doctor without the supervision of the team leader, while other steps in the surgery may be performed while the team leader is present in the operating room supervising the resident.

Some hospitals may use a single surgeon to supervise a number of resident doctors that are performing portions of the procedures during concurrent surgeries.

### SUMMARY

Embodiments of the present invention are directed to a computer-implemented method for surgical scheduling. A non-limiting example of the computer-implemented method includes receiving a surgical protocol for a surgical procedure, a team member identifier for a team member, and team member data associated with a plurality of steps in the surgical protocol. The method further includes determining, by a processing device, a number of times n that the team member has performed a particular step of the plurality of steps of the surgical protocol at an unassisted qualification level. The method further includes determining, by the processing device, whether the number of times n meets a threshold level. The method further includes, responsive to determining that the number of times n does not meet the threshold level, designating the particular step as having an observation requirement by a team leader. The method further includes, responsive to determining that the number of times n meets the threshold level, designating the particular step as having no observation requirement by the team leader. The method further includes presenting, on a display, the plurality of steps in the surgical protocol to the team member, wherein the particular step of the plurality of steps comprises an indicator that corresponds to one of having the observation requirement or having no observation requirement.

Embodiments of the present invention are directed to a system. A non-limiting example of the system includes a memory comprising computer readable instructions and a processing device for executing the computer readable instructions for performing a method for generating a surgical protocol. A non-limiting example of the method includes receiving, by the processing device, an input indicative of a team leader threshold for a number of times a team member performs a step of a surgical protocol at an unassisted qualification level. The method further includes generating, by the processing device, the surgical protocol. The method further includes adding, by the processing device, a step to the surgical protocol. The method further includes saving, by the processing device, the surgical protocol in the memory.

Embodiments of the invention are directed to a computer program product. A non-limiting example of the computer program product includes a computer readable storage medium having program instructions embodied therewith. The program instructions are executable by a processor to cause the processor to perform a method for generating a step in a surgical procedure. A non-limiting example of the method includes receiving, by the processing device, an input comprising a step name of a step of a plurality of steps. The method further includes receiving, by the processing device, a description associated with the step. The method further includes receiving, by the processing device, media associated with the step. The method further includes associating, by the processing device, the step with the surgical protocol. The method further includes saving, in the memory, the step name, the description, the surgical protocol, and the media associated with the step.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

FIG. 1 illustrates a block diagram of an example surgical scenario.

FIG. 2 illustrates a block diagram of an exemplary embodiment of a processing system.

FIGS. 3A and 3B illustrate a block diagram of an exemplary method that may be performed by the processing device (of FIG. 2).

FIG. 4 illustrates a block diagram of a method for generating a surgical protocol using the system described herein.

FIG. 5 illustrates a block diagram of an exemplary method for generating a step in a protocol.

### DETAILED DESCRIPTION

As discussed above, some hospitals may use a single surgeon to supervise a number of resident doctors that are performing portions of different surgeries, in different operating rooms, concurrently. During such concurrent surgical procedures, each resident doctor may perform different steps of a surgical protocol with varying degrees of proficiency, and with varying degrees of supervision from the surgeon.

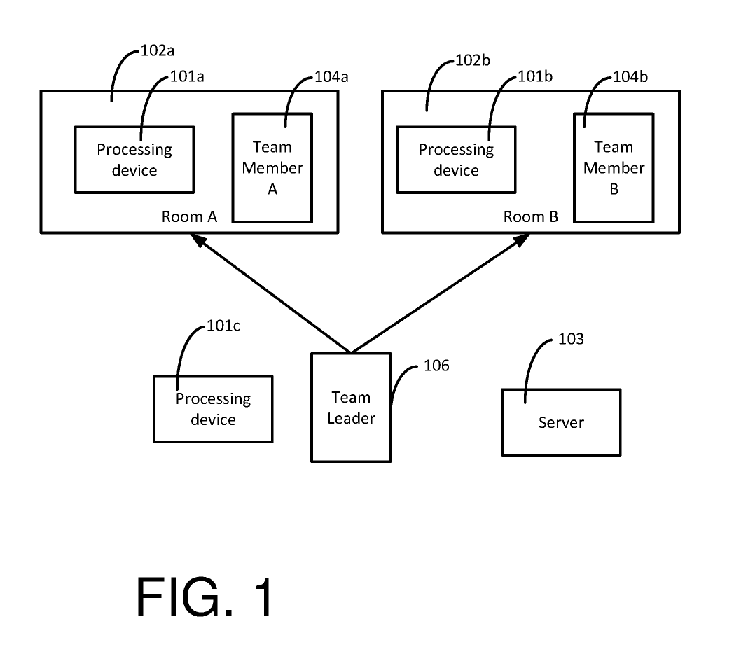

FIG. 1 illustrates a block diagram of an example surgical scenario. In the example surgical room A 102a is occupied by team member A 104a who is a resident doctor (surgeon in training) and surgical room B 102b is occupied by a team member B 104b who is a resident doctor (surgeon in training).

Each of the team members A and B 102a and 102b are supervised by a team leader (qualified surgeon) 106.

The team members A and B 102a and 102b may be performing similar or different surgical protocols (surgical procedures) that each include a number of steps. Each step of each protocol may have varying levels of difficulty, and the team members A and B 102a and 102b have different levels of experience performing the steps of each protocol.

In practice, it is desirable for the team leader 106 to be present in the surgical room 102a or 102b during challenging steps, or during particular steps with a particular team member 104a or 104b who has limited experience performing a particular step. Once a team member A or B 104a or 104b becomes proficient in performing a particular step, the team leader 106 need not be present in the surgical room 102a or 102b while the team member performs the step.

Processing devices 101a, 101b, and 101c are communicatively connected, and may include any type of terminal including a personal computer, a display, touch screen display, tablet, or smartphone. The processing device 101a is located in the surgical room A 102a, the processing device 101b is located in the surgical room B 102b, and the processing device 101c is located with or proximate to the team leader 106. In some exemplary embodiments, the processing devices 101a, 101b, and 101c may communicate directly using a network such as an ad hoc network. In other exemplary embodiments, the processing devices 101a, 101b, and 101c may share and exchange information and data via a server 103 or other type of suitable processing and communication system.

The systems and methods described herein provide data and guidance to the surgical teams while performing any number of surgeries concurrently or substantially simultaneously.

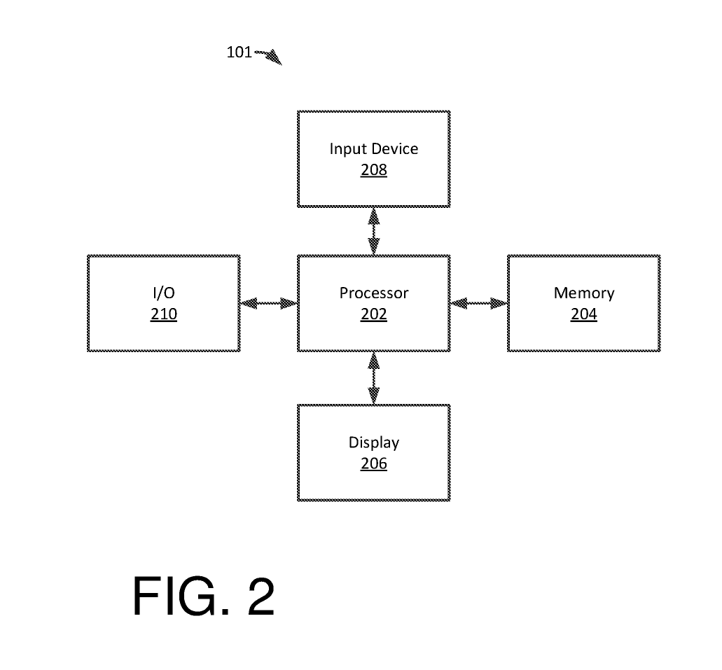

FIG. 2 illustrates a block diagram of an exemplary embodiment of a processing device 101. The device 101 includes a processor 202 that is communicatively connected to a memory 204, a display 206, an input device 208, and an input/output (I/O) portion 210 that is operative to be connected to a network such as, a local area network or a wide area network.

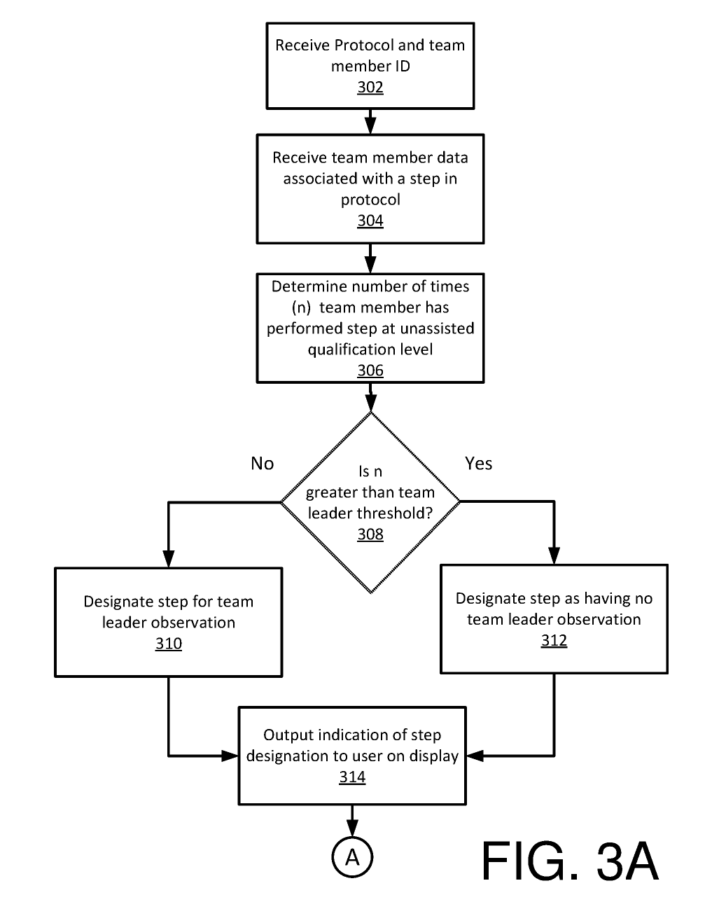

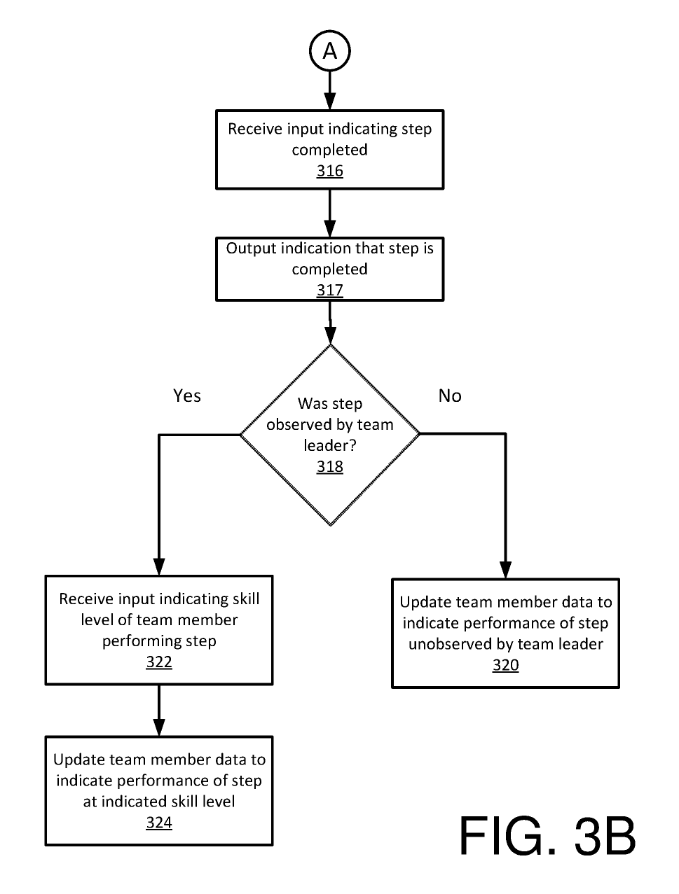

FIGS. 3A and 3B illustrate a block diagram of an exemplary method that may be performed by the processing device 101 (of FIG. 2). In block 302 a protocol is received and team member identifications are received. The team member identifiers are created when the team member creates an account or profile and are unique to the team member.

A protocol includes a list of steps that are performed in a surgical procedure. Each protocol has a unique identifier. Each step may include a step name or identifier and a description of how to perform the step, which may include media such as audio or video media.

The team member identifications (ID) identify the team members (e.g., team member A 104a and team leader 106 of FIG. 1) that will perform the received protocol.

In block 304, team member data associated with the steps in the protocol are received. The team member data includes the number of times a team member has performed a particular step in the protocol at a particular skill level.

In block 306, the system determines the number of times (n) a team member has performed a particular step in the protocol at an unassisted skill level using the received team member data.

The system determines whether n is greater than a threshold level of the team leader 106 in block 308. In this regard, the team leader 106 (qualified surgeon) has entered a threshold level for a number of times that a team member 104a should perform any step at an unassisted skill level before the team member may perform the step without the presence of the team leader 106 in the surgical room 102a.

If n is not greater than the team leader threshold, the step is designated for team leader observation or presence in block 310. If n is greater than the team leader threshold, the step is designated as having no team leader observation in block 312.

In block 314, the step or steps in the protocol are presented to the user on a display where the steps are listed with indicators that correspond to the step designation for the particular step. In this regard, the display may show the protocol with a listing of each of the steps in the protocol. Each of the steps has an associated textual or non-textual indicator such as, for example, an icon, a sound, a vibration, a color code that indicates the designation of the particular step.

Referring now to FIG. 3B, in block 316, the system receives an input indicating that a step has been completed. The input may include, for example, a user input to the device 101. In block 317, the system outputs an indication that the step has been completed.

In block 318 the system determines whether the step was designated for team leader 106 observation. If no, in block 320, the team member data is updated to indicate that the team member 104a performed the step unobserved by the team leader 106. If yes, in block 322 the system receives an input indicating the skill level of the team member performing the step. The team member data is updated to indicate the performance of the step at the indicated skill level in block 324.

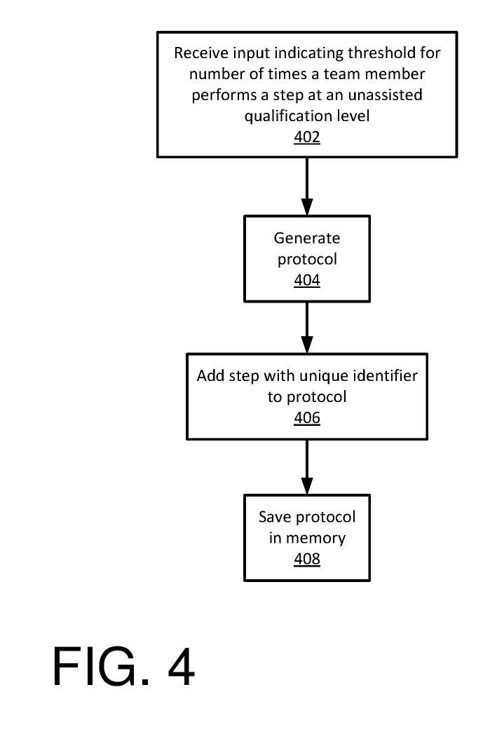

FIG. 4 illustrates a block diagram of an exemplary method for generating a surgical protocol using the system described herein. In block 402, an input is received that indicates a team leader threshold for a number of times a team member performs a step at an unassisted qualification level.

In block 404, a surgical protocol is generated. The surgical protocol has a unique identifier such as a name or identification number and may be associated with an identifier of the author of the surgical protocol.

In block 406, a step is added to the protocol. Each step added to the protocol has unique identifiers that may include names or numbers. In block 408, the protocol is saved in memory.

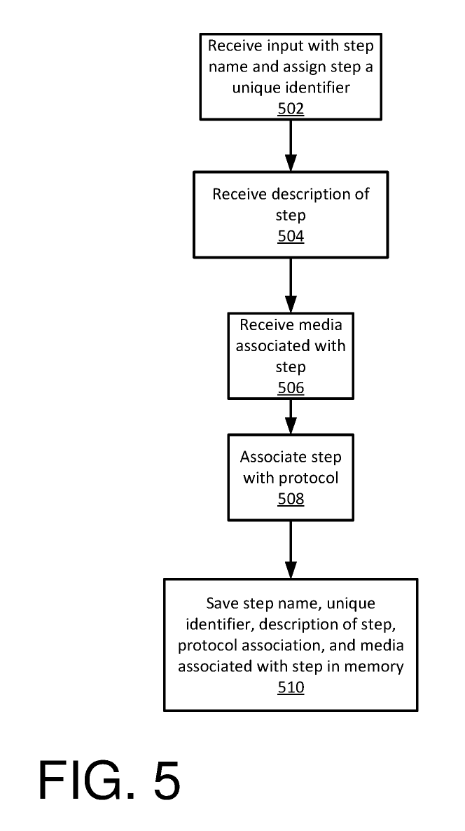

FIG. 5 illustrates a block diagram of an exemplary method for generating a step in a protocol. In block 502, the system receives an input with a step name. The user or the system may generate a unique identifier for the step. In block 504, a description of the step is received. The description of the step may include, for example, an explanation of the step, or a listing of sub-steps that are performed to complete the step. Media such as, for example, video, visual, photographic, graphical or audio associated with the step is received in block 506.

The step is associated with a protocol in block 508. In block 510 the step name, unique identifier, description of the step, associated protocol, and media associated with the step are saved in memory.

The systems and methods described herein provide for a system where a qualified surgeon may supervise any number of surgical protocols that are being performed simultaneously or concurrently in different operating rooms. The system is operable to display each of the steps of the protocols that are being performed. The system indicates which steps, based on the experience and skill level of the resident surgeons, should be observed by the qualified surgeon, and which steps may be performed by the resident surgeons without the observation of the qualified surgeon.

Such a system improves the safety and efficiency of the surgical teams by tracking the skill levels of the resident surgeons with respect to particular steps in the surgical protocols. By tracking the skill levels and experiences of the resident surgeons, the system identifies and outputs to the surgical teams which steps for each protocol should be observed by the qualified surgeon.

What is claimed is:

1. A computer-implemented method for surgical scheduling, the method comprising:
receiving a surgical protocol for a surgical procedure, a team member identifier for a team member, and team member data associated with a plurality of steps in the surgical protocol;
determining, by a processing device, a number of times n that the team member has performed a particular step of the plurality of steps of the surgical protocol at an unassisted qualification level;
determining, by the processing device, whether the number of times n meets a threshold level;
responsive to determining that the number of times n does not meet the threshold level, designating the particular step as having an observation requirement by a team leader;
responsive to determining that the number of times n meets the threshold level, designating the particular step as having no observation requirement by the team leader; and
presenting, on a display, the plurality of steps in the surgical protocol to the team member, wherein the particular step of the plurality of steps comprises an indicator that corresponds to one of having the observation requirement or having no observation requirement.

2. The computer-implemented method of claim 1, wherein the threshold level is assigned by the team leader, the team leader being a qualified surgeon.

3. The computer-implemented method of claim 1, wherein the threshold level is a number of times that the team member should perform the particular step of the plurality of steps of the surgical protocol at an unassisted skill level before the team member performs the particular step without the team leader being present.

4. The computer-implemented method of claim 1, wherein the indicator is a textual indicator that indicates the designation of the particular step as one of having the observation requirement or having the no observation requirement.

5. The computer-implemented method of claim 1, wherein the indicator is a non-textual indicator that indicates the designation of the particular step as one of having the observation requirement or having no observation requirement.

6. The computer-implemented method of claim 1, further comprising:
receiving an input indicating that a step of the plurality of steps is completed; and
presenting, on the display, a completion indication that indicates that the step of the plurality of steps is a completed step.

7. The computer-implemented method of claim 6, further comprising:
determining, by the processing device, whether the completed step was designated as having one of the observation requirement and the no observation requirement.

8. The computer-implemented method of claim 7, further comprising:
responsive to determining that the completed step was designated as having the observation requirement, receiving an input indicating a skill level of the team member performing the completed step; and
updating the team member data for the team member to indicate the performance of the completed step with observation by the team leader.

9. The computer-implemented method of claim 7, further comprising:
responsive to determining that the completed step was designated as having the no observation requirement, updating the team member data for the team member to indicate the performance of the completed step without observation by the team leader.

10. A system comprising:
a memory comprising computer readable instructions; and
a processing device for executing the computer readable instructions for performing a method for generating a surgical protocol, the method comprising:
receiving, by the processing device, an input indicative of a team leader threshold for a number of times a team member performs a step of a surgical protocol at an unassisted qualification level;
generating, by the processing device, the surgical protocol;
adding, by the processing device, a step to the surgical protocol; and
saving, by the processing device, the surgical protocol in the memory.

11. The system of claim 10, wherein the surgical protocol comprises a unique identifier and is associated with an identifier of an author of the surgical protocol.

12. The system of claim 10, wherein the step added to the protocol comprises a unique identifier.

13. A computer program product comprising:
a computer readable storage medium having program instructions embodied therewith, the program instructions executable by a processing device to cause the processing device to perform a method for generating a step in a surgical protocol, the method comprising:
receiving, by the processing device, an input comprising a step name of a step of a plurality of steps;
receiving, by the processing device, a description associated with the step;
receiving, by the processing device, media associated with the step;
associating, by the processing device, the step with the surgical protocol; and
saving, in the memory, the step name, the description, the surgical protocol, and the media associated with the step.

14. The computer program product of claim 13, the method further comprising generating a unique identifier for the step.

15. The computer program product of claim 13, wherein the description of the step comprises an explanation of the step.

16. The computer program product of claim 13, wherein the description of the step comprises a listing of sub-steps that are performed to complete the step.

17. The computer program product of claim 13, wherein the media comprises at least one of a video, a visual, a graph, a photograph, and audio.
